(19) United States
(12) Patent Application Publication
(10) Pub. No.: US 2018/0296196 A1
(43) Pub. Date: Oct. 18, 2018

(54) METHOD AND DEVICE FOR COLLECTING MYCOBACTERIUM COMPLEX SPECIES FROM TEH ORAL CAVITY OF A PATIENT

(71) Applicants: NORTH-WEST UNIVERSITY, Potchefstroom (ZA); STELLENBOSCH UNIVERSITY, Stellenbosch (ZA); UNIVERSITY OF NEBRASKA-LINCOLN, Lincoln, NE (US)

(72) Inventors: Anne Frederica GROBLER, Potchefstroom (ZA); Lizl CRONJE, Stellenbosch (ZA); Hendrik VILJOEN, Lincoln, NE (US)

(21) Appl. No.: 15/578,842

(22) PCT Filed: Jun. 1, 2016
(86) PCT No.: PCT/IB2016/053212

§ 371 Date: Dec. 1, 2017

(30) Foreign Application Priority Data
Jun. 1, 2015 (ZA) 201503932

(51) Int. Cl. A61B 10/00; A61J 17/00; G01N 33/569

(52) U.S. Cl. CPC A61B 10/0051; A61B 2010/0216; G01N 33/5695; A61J 17/001

(57) ABSTRACT

This invention relates to a method and device for collecting a Mycobacterium complex species, such Mycobacterium tuberculosis (MTB) from the oral cavities of particularly children and infants. A device is provided which has a distal end and an operative end portion manufactured from a parent polymer which has been functionalised with a chemical compound having an affinity to the Mycobacterium complex species to collect same such that the presence thereof in the sample can be detected. The invention further extends to a method of preparing biological material obtained from such collected sample.

### FIELD OF THE INVENTION

This invention relates to a method and device for collecting Mycobacterium complex species, particularly, but not exclusively, to a method and device for collecting Mycobacterium tuberculosis (MTB) from the oral cavities of children and infants. The invention further relates to a method of preparing biological material obtained from such collected sample.

### BACKGROUND TO THE INVENTION

Tuberculosis (TB) is a disease caused by MTB and is responsible for approximately 1.4 million deaths annually and it disproportionately affects developing countries, particularly sub-Saharan Africa. Notwithstanding technical advances, expanded programs and significant expenditures, the disease is still not under control.

Despite being a preventable and treatable disease, TB remains a leading cause of morbidity and mortality worldwide. It is estimated that one third (2.9 million) of all TB cases are not notified to the health systems. This gap is in part due to missed diagnosis; diagnostics account for only 2% of the cost of health care, but affect 60-70% of treatment decisions [1].

The current methods and tools for detecting TB are focussed on the adult population. These diagnostics include antibody based methods, PCR based methods using sputum samples, bronchial washings and urine samples. Some additional methods include mass spectrometry to detect mycolic acids, and a gas sensor to detect different species of Mycobacteria. Some devices for mouth swabs have also been proposed, but without apparent success and/or efficacy.

Despite the existing means alluded to above, there remains a lack of a sufficiently sensitive tool to detect early disease in adults, paucibacillary disease in HIV infected adults and TB in all children. Young children and HIV infected adults are particularly difficult to diagnose; obtaining adequate sputum from young children requires relatively invasive and resource-intensive procedures.

Children respond differently to a TB challenge than adults; their immunological response is not the same and the disease presents with different pathology. The lymph plays a key role in the immunological response and it is conceivable that bacilli (free or phagocytised) may traffic via the lymphatic system [2]. Children with TB typically present with enlarged mediastinal and hilar lymph nodes. An example of this patho-physiological response is scrofula. The salivary glands, particularly the parotid, have embedded lymph nodes; nodes are also located close to the submaxilliary gland.

The MTB bacilli may also be present in the oral cavity as a result of transfer from the lungs into the oral cavity through coughing or sneezing. The collection of salivary specimens from the oral cavity to test for the presence of bacilli is accordingly a procedure for the diagnosis of tuberculosis and may also contribute to existing knowledge of TB pathology in children.

Current methods to collect respiratory specimens for TB investigation from young children are relatively invasive, including procedures such as gastric aspiration, sputum induction with nasopharyngeal suctioning or bronchoalveolar lavage. In many settings the diagnosis is made clinically, oftentimes by nurses or doctors not necessarily trained in paediatric TB.

The accurate diagnosis of TB in young children requires in addition that the specimen collection methods are also improved in order to provide samples with adequate bacteriological yield. As most of the laboratory techniques used for the diagnosis of TB are dependent on the detection of MTB, the pathogen responsible for the disease, it is of utmost importance that good quality specimens are obtained with the highest possible concentration of Mycobacteria to enable the accurate diagnosis of TB. In the current era of multi drug-resistant (MDR)/extensively drug-resistant (XDR)/totally drug-resistant (TDR)-TB, there is also a growing need to isolate the bacillus in order to perform drug susceptibility testing. It is therefore vital to improve the specimen collection method in children in order to provide samples with adequate bacteriological yield for fast and accurate detection and timely treatment.

New TB diagnostics, which are simple, sensitive, rapid and which use culturally acceptable biological samples, are crucial to curb the global TB epidemic.

### OBJECT OF THE INVENTION

It is accordingly an object of the current invention to provide a method and device for collecting Mycobacterium complex species, such as Mycobacterium tuberculosis with which the abovementioned disadvantages could at least partially be alleviated or overcome and to provide a method of preparing biological material from such collected sample.

### SUMMARY OF THE INVENTION

According to a first aspect of the invention there is provided a device for collecting a biological sample from the oral cavity of a patient comprising a body having a distal end and an operative end portion, at least the operative end portion being manufactured from a parent polymer which has been functionalised with a chemical compound having an affinity to a Mycobacterium complex species to collect and operatively detect the presence of a Mycobacterium complex species in the sample.

The invention provides for the parent polymer to be selected from any one or more of the group consisting of silicon based polymers, polyester based polymers and polyurethane based polymers.

The invention yet further provides for the chemical compound to be provided as either one or more of Concanavalin A and a positive charged ammonium moiety and a hydrocarbon chain; and wherein the parent polymer is functionalised by attaching the Concanavalin A and the positive charged ammonium moiety and hydrocarbon chain to the parent polymer covalently or electrostatically. The hydrocarbon chain may be provided as a C₁₀, C₁₂ or C₁₄ hydrocarbon chain.

The operative end portion may be in the form of a pacifier or a brush having bundled strands.

The distal end may be in the form of a handle.

The invention provides for the Mycobacterium complex species to include Mycobacterium tuberculosis.

The invention yet further provides for the patient to include a child or infant.

According to a second aspect of the invention there is provided a method of collecting a Mycobacterium complex species present in a biological sample from the oral cavity of a patient comprising:
providing a parent polymer;
functionalising the parent polymer with a chemical compound having an affinity to the Mycobacterium complex species;
bringing the functionalised polymer into contact with the inside walls of the oral cavity of the patient; and
removing said functionalised polymer from the oral cavity of the patient.

The parent polymer may be selected from any one or more of the group consisting of silicon based polymers, polyester based polymers and polyurethane based polymers.

The step of functionalising the parent polymer may include the step of attaching the chemical compound to the parent polymer covalently or electrostatically.

The parent polymer may be functionalised with the chemical compound being either one or more of Concanavalin A or a positive charged ammonium moiety and a hydrocarbon chain; and wherein the Concanavalin A and the positive charged ammonium moiety and hydrocarbon chain is attached to the parent polymer covalently or electrostatically. The hydrocarbon chain may be provided as a C₁₀, C₁₂ or C₁₄ hydrocarbon chain.

The invention provides for adding a flavouring agent to the polymer to make collecting the biological sample more acceptable to the patient.

In accordance with a third aspect of this invention, there is provided the use of the device or method, as described hereinbefore, for collecting a biological sample having a Mycobacterium complex species from the oral cavity of a patient for diagnosis of Tuberculosis.

In accordance with a fourth aspect of this invention, there is provided a method for collecting Mycobacterium complex species present in a biological sample and preparing biological material therefrom comprising the steps of:
A. collecting the Mycobacterium complex species as described hereinbefore to form biological material characterised in that the functionalised polymer forms a capturing scaffold; and
B. preparing the biological material including the steps of altering the constitutive characteristics of the sample in the presence of the capturing scaffold by concomitantly:
   adding a lysis buffer containing a solubilising agent, a detergent and preferably a reducing agent to the sample;
   and concomitantly physically treating the biological sample through agitation and elevating the temperature of the biological sample above 40 degrees Celsius and up to 100 degrees Celsius, preferably 92 degrees Celsius;
for simultaneously:
   inhibiting coagulation of the sample;
   lysing the sample to release the biological material from the sample, thus making the biological material available; and
   capturing at least one fraction of the biological material on the capturing scaffold.

Subsequently the capturing scaffold, with the particular fraction of biological material captured thereon, is removed from the remainder of the sample and preferably washed with de-ionised water. The capturing scaffold is subsequently stored, transported or presented for analysis of the biological sample.

According to a fifth aspect of the invention there is provided a kit for use in a method of collecting Mycobacterium complex species present in a biological sample and preparing biological material therefrom, the kit comprising:
a device for collecting the biological sample as described hereinbefore, the device forming a capturing scaffold; and
a container including a lysis buffer containing a solubilising agent, a detergent and preferably a reducing agent for operatively simultaneously lysing the biological sample to make the biological sample available, inhibiting coagulation of the biological sample, and capturing at least one fraction of the biological material on the capturing scaffold.

### BRIEF DESCRIPTION OF THE ACCOMPANYING DRAWINGS

The invention is now described by way of non-limiting examples with reference to the accompanying drawings wherein:

FIG. 1: is a perspective view of a device according to a first embodiment of the invention;

FIG. 2: is a perspective view of a device according to a second embodiment of the invention; and

FIG. 3: is a fluorescence microscopy image showing captured bacilli visible on the surface of a functionalised parent polymer.

### DESCRIPTION OF PREFERRED EMBODIMENTS OF THE INVENTION

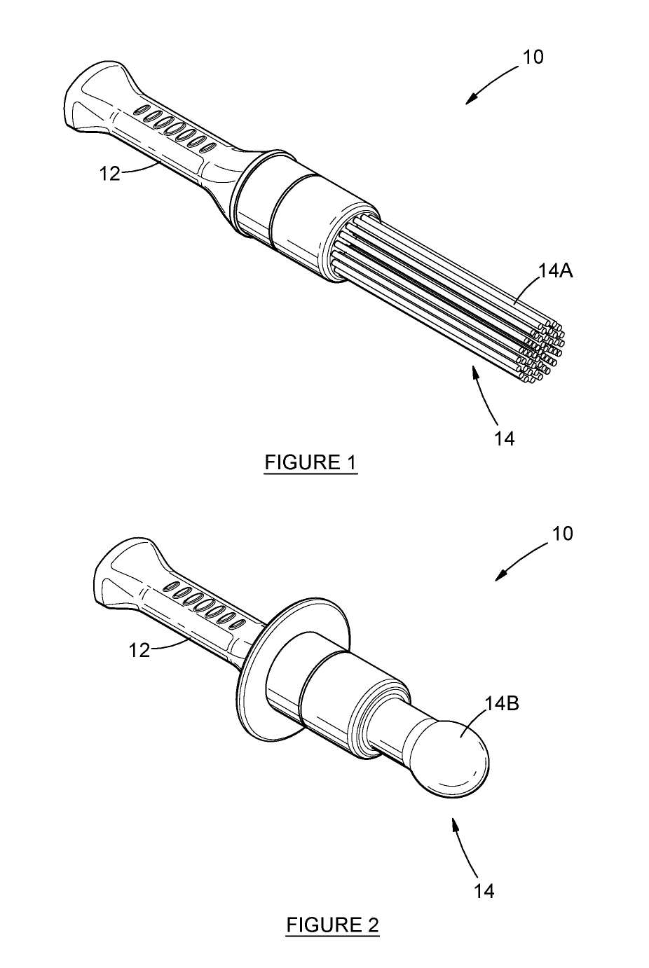

Referring to FIG. 1, a device according to a first preferred embodiment of the invention, for collecting Mycobacterium tuberculosis (MTB) from the oral cavities of children and infants, is generally designated by numeral 10.

The device 10 has a body provided with a distal end in the form of a handle 12 which can be manipulated by the user during use as will be explained below; and an operative end portion 14 which, in the embodiment shown in FIG. 1, is in the form of bundled strands of fibres flared into a brush 14A.

In the alternative embodiment shown in FIG. 2, the operative end portion 14 is in the form of a pacifier 14B.

The brush 14A and pacifier 14B consists of a parent polymer functionalised with a variety of chemical compounds/ligands to aid the binding/capture of microbes, more specifically mycobacteria and even more specifically Mycobacterium tuberculosis (MTB). Two chemical compounds/ligands are used namely Concanavalin A (a carbohydrate-binding protein) and a chemical compound with a positive charged ammonium moiety and a hydrocarbon chain (“ConA” and “Cx”). It will be understood that the Cx may include a C₁₀, C₁₂ or C₁₄ hydrocarbon chain.

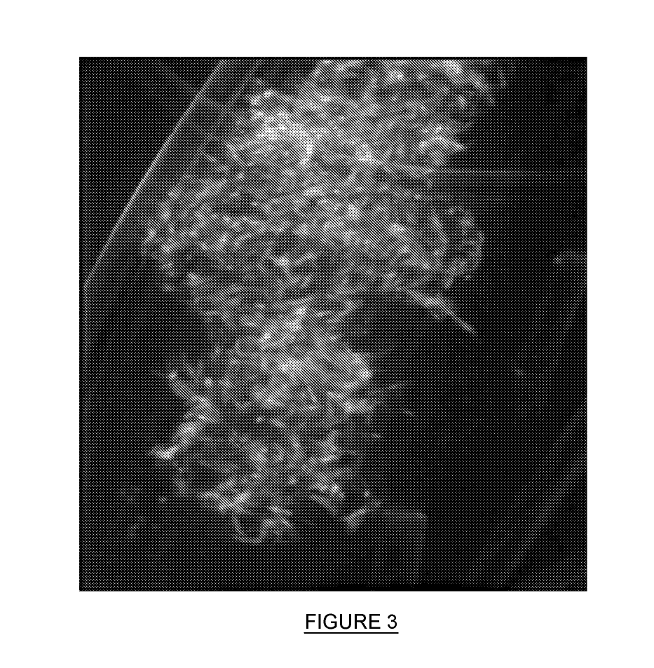

FIG. 3 illustrates the fluorescence microscopy image of a modified polymer (functionalised with Cx), in this instance using the parent polymer styrene-co-maleic anhydride (SMA), after incubation with an attenuated mycobacterial strain at 37° C. for one hour. The captured bacilli are visible on the surface of the modified polymer.

For the brush 14A, a modified polyester-based polymer will be used as parent polymer, functionalised with ConA and Cx. The brush is injection moulded and the handle 12 is accordingly made from the functionalised polymer, although the handle 12 can be made of any other suitable plastic material.

Pacifiers 14B are manufactured using silicone- or polyester-based polymers that are safe, non-toxic and inert. The pacifiers 14B will be functionalised with the two chemical compounds mentioned previously. The pacifiers 14B are injection moulded using the functionalised polymer.

Both ConA and Cx functionalisation agents will be attached to the parent polymer covalently or electrostatically to prevent leaching when applied to the environment in the oral cavity.

Flavouring may also be added to the polymer which will make the collection process more acceptable to patients.

In use, the device 10 is simply inserted into the oral cavity to bring the device into contact with the inside walls of the oral cavity of the patient and removed therefrom to collect a biological sample for further preparation and processing.

The samples are further prepared, processed and analysed by smear microscopy, culture, GeneXpert or any other suitable method known in the art to detect the presence of Mycobacterium complex species. Preparation of the biological sample will specifically by done by the molecular assay as described in the co-applicant's PCT/IB2012/054608, the contents of which is incorporated fully herein. The device 10 can be fitted directly into the the lysis micro reactor (‘LMR’) or processing device (not shown) disclosed by PCT/IB2012/054608 and the handle 12 furthermore is moulded to a diameter that will serve as a lid for the mentioned LMR device. In this instance, the operative end portion 14, forms the capturing scaffold as described below.

Preparing biological material from the biological sample in the form of the sputum sample obtained by the steps mentioned above will now be described.

The method of preparing biological material includes the steps of altering the constitutive characteristics of the sample in the presence of the capturing scaffold by concomitantly:
adding a lysis buffer containing a solubilising agent, a detergent and a reducing agent to the sample;
elevating the temperature of the sample above 40 degrees Celsius and up to a 100 degrees Celsius, preferably 92 degrees Celsius; and
physically treating the sample through agitation,
   for simultaneously:
inhibiting coagulation of the sample;
lysing the sample to release the biological material from the sample, thus making the biological material available; and
capturing at least one fraction of the biological material on the capturing scaffold.

Subsequently the capturing scaffold, with the particular fraction of biological material captured thereon, is removed from the remainder of the sample and washed with de-ionised water. The capturing scaffold is subsequently stored, transported or presented for analysis of the biological sample.

Purified starch such as purified corn or potato starch is optionally added to the biological material simultaneously with the lysis buffer for neutralising any FOR inhibitors that may be present in the biological material.

The solubilising agent comprises a chaotropic salt in the lysis buffer. The lysis buffer is selected from the group consisting of phosphate buffers, 4-(2-hydroxyethyl)-1-piperazineethanesulfonic acid (HEPES), N-Cyclohexyl-2-aminoethanesulfonic acid (CHES), N-cyclohexyl-3-aminopropanesulfonic acid (CAPS) and piperazine-N,N-bis(2-ethanesulfonic acid) (PIPES), Tris-HCl, as well as other tris(hydroxymethyl)aminomethane (Tris) buffers containing ethylene diamine tetra-acetic acid (EDTA), ethylene glycol tetra-acetic acid (EGTA), deoxycholate, sodium chloride (NaCl), sodium phosphate, octylphenoxypolyethoxyethanol, and non-ionic surfactants provided with a hydrophilic polyethylene oxide group and a hydrocarbon lipophilic or hydrophobic group, and combinations thereof. The lysis buffer has a pH of between 4 and 9, preferably 7.5.

The chaoropic salt is selected from the group consisting of urea, thiourea, guanidine hydrochloride, lithium perchlorate, sodium iodine, sodium perchlorate, guanidine isothiocyanate, guanidine carbonate, guanidine thiocyanate, derivatives thereof, preferably guanidine hydrochloride and combinations thereof.

The detergent is selected from the group consisting of 3-[(3-cholamidopropyl) dimethylammonio]-1-propanesulfonate (CHAPS), a nonionic surfactant and emulsifier derived from polyethoxylated sorbitan and oleic acid, a nonionic surfactant which has a hydrophilic polyethylene oxide group and a hydrocarbon lipophilic or hydrophobic group, saponin, sodium deoxycholate, SDS, octyl glucoside, octyl thioglucoside, laurly maltose, octylphenoxypolyethoxyethanol, and combinations thereof.

The reducing agent is selected from the group consisting of 2-mecarptoethanol, dithiothreitol (DTI), 2-mercaptoethylamine, Tris(2-carboxyl)phosphine (TCEP), cysteine HCl, N-ethylmaleimide, Nacystelyn, dornase alfa, thymosin β₄, guaifenesin TCEP HCl, and combinations thereof.

The prepared biological material captured on the capturing scaffold is subsequently air dried and stored at ambient temperature. The capturing scaffold is stored, transported or presented for analysis of the biological sample.

The invention accordingly provides a functionalised polymer which specifically binds to mycotic acids, the hallmark component of mycobacterial cell walls. It also utilizes the negative charge of the mycobacterial cell wall to bind electrostatically via the positive charge on the polymer backbone.

Collecting clinical specimens from the oral cavities of infants and children with either the brushes 14A or pacifiers 14B as described for the diagnosis of TB is accordingly a viable, less invasive alternative to current methods and provides a feasible approach for collecting biological samples at home by parents or at schools and clinics under supervision of teachers and nurses.

The use of the invention to scavenge the oral cavity makes the process less invasive, more accessible and less stigmatising to the broader public. In the case of infants, the use of a pacifier is proposed.

The use of brushes and pacifiers that is made of a material that has high affinity for binding specifically MTB complex and which is safe for use in humans is accordingly proposed. This has never been tested or suggested as a diagnostic procedure for TB. This method and device for specimen collection would accordingly remove a major barrier to TB diagnosis in children, by avoiding the need for time-consuming, resource-intensive and relatively invasive procedures for sputum collection or obtaining gastric aspirates.

The invention also provides for a method and device which provides biological samples with adequate bacteriological yield. The capturing of the mycobacteria on a limited surface acts as a concentration step for mycobacteria in a sample.

Such a concentration step increases the number of mycobacteria/volume of sample obtained.

Yet further, for sputum-scarce adults, brushings collected from the oral cavity may also aid in improving diagnosis. The simplicity of this invention would enable the collection technique to be rolled out at point-of-care and even community level. It could potentially become an effective screening test for the early detection of TB disease, and it could have major impact on global TB control.

A simple, non-invasive specimen collection method coupled with a highly sensitive assay results in early detection of TB disease and will accordingly impact on TB transmission in communities as well as reduce TB-related morbidity and mortality among children, who are most difficult to diagnose using current available methods. The impact will broaden significantly if the system performs well with adults—this specimen collection method will be non-stigmatizing and enjoy better cultural acceptance, and it could open the way to screening for TB.

The invention provides a simple, culturally acceptable and highly sensitive tool to detect early TB disease in adults and diagnose paucibacillary disease in children.

This tool is also robust, safe for use by the patient and caregiver, as well as cost effective, since TB is a disease of the developing world.

The invention yet further provides an efficient and robust method for preparing uncontaminated biological material from the collected sputum sample without applying numerous consecutive steps that necessarily have to be taken in a laboratory environment or in different chambers of the LMR. It was found that, in particular, a kit comprising the said capturing scaffold strip and lysis buffer as described above could be prepared and provided to field workers collecting samples in rural areas using the LMR. The sample could be combined with the scaffold and lysis buffer in the LMR and the scaffold strip removed once the biological material has been captured on the scaffold strip. The advantages of this single step method over the relatively complex prior art methods requiring a laboratory environment, numerous enzymes and other reagents and multiple steps, are evident.

It is accordingly asserted that the disadvantages associated with known methods and devices for collecting Mycobacterium complex species could be alleviated with the method and device according to the invention.

It will be understood that the above examples are not to be interpreted as limiting the scope of the invention. For example, it must be understood that the use of functionalised polymers to capitalise on specific moieties expressed on cell exteriors is not limited to the above example and may find wider application without departing from the scope of the invention. The Mycobacterium complex species may furthermore include those associated with Leprosy disease.

Similarly, the mentioned parent polymers and chemical compounds having affinity to Mycobacterium is not to be limited to those mentioned herein.

### REFERENCES

[1] BRINGING THE LAB TO THE PATIENT: developing point-of-care diagnostics for resource limited settings, A REPORT FROM THE AMERICAN ACADEMY OF MICROBIOLOGY. 2012.
[2] Behr, M A and W R Waters, Is tuberculosis a lymphatic disease with a pulmonary portal? www.thelancet.com/infection Published online Nov. 22, 2013 http://dx.doi.org/10.1016/S1473-3099(13)70253-6.

1. A device for collecting a biological sample from the oral cavity of a patient, comprising a body having a distal end and an operative end portion, at least the operative end portion being manufactured from a parent polymer which has been functionalised with a chemical compound having an affinity to a Mycobacterium complex species to collect and operatively detect the presence of a Mycobacterium complex species in the sample.

2. The device according to claim 1, wherein the parent polymer is selected from the group consisting of a silicon based polymers, a polyester based polymers and a polyurethane based polymers.

3. The device according to claim 1, wherein the parent polymer has been functionalised with one or more of Concanavalin A and a positive charged ammonium moiety and a hydrocarbon chain.

4. The device according to claim 3, wherein the Concanavalin A and the positive charged ammonium moiety and hydrocarbon chain are attached to the parent polymer covalently or electrostatically.

5. The device according to claim 3, wherein the hydrocarbon chain is a C₁₀, C₁₂ or C₁₄ hydrocarbon chain.

6. The device according to claim 1, wherein the operative end portion is in the form of a pacifier or a brush having bundled strands.

7. The device according to claim 1, wherein the distal end is in the form of a handle.

8. The device according to claim 1, wherein the Mycobacterium complex species comprises Mycobacterium tuberculosis.

9. The device according to claim 1, wherein the patient is a child or infant.

10. A method for collecting a Mycobacterium complex species present in a biological sample from the oral cavity of a patient, comprising:
providing a parent polymer;
functionalising the parent polymer with a chemical compound having an affinity to said Mycobacterium complex species;
bringing the functionalised polymer into contact with the inside walls of the oral cavity of the patient; and
removing said functionalised polymer from the oral cavity of the patient.

11. The method according to claim 10, wherein the parent polymer is selected from the group consisting of a silicon based polymers, a polyester based polymers and a polyurethane based polymers.

12. The method according to claim 10, wherein the chemical compound is one of Concanavalin A and a positive charged ammonium moiety and a hydrocarbon chain.

13. The method according to claim 12, wherein the parent polymer has been functionalised by attaching the Concanavalin A and the positive charged ammonium moiety and hydrocarbon chain to the parent polymer covalently or electrostatically.

14. The method according to claim 12, wherein the hydrocarbon chain is a C₁₀, C₁₂ or C₁₄ hydrocarbon chain.

15. The device of claim 1, comprising adding a flavouring agent to the parent polymer to make collecting the biological sample more acceptable to the patient.

16. (canceled)

17. A method for collecting Mycobacterium complex species present in a biological sample and preparing biological material therefrom, comprising:
(a) collecting the Mycobacterium complex species according to claim 10 to form a biological material characterised in that the functionalised polymer forms a capturing scaffold; and
(b) preparing the biological material altering the constitutive characteristics of the sample in the presence of the capturing scaffold by concomitantly:
adding a lysis buffer containing a solubilising agent, and a detergent;
and concomitantly physically treating through agitation and elevation of the temperature of the biological sample above 40 degrees Celsius and up to 100 degrees Celsius, for simultaneously:
inhibiting coagulation of the sample;
lysing the sample to release the biological material from the sample, and make the biological material available; and
capturing at least one fraction of the biological material on the capturing scaffold.

18. The method according to claim 17, further comprising removing the capturing scaffold, with the fraction of biological material captured thereon, from the remainder of the sample.

19. The method according to claim 18, further comprising storing, transporting or presenting the biological sample for analysis.

20. A kit for use in a method of collecting Mycobacterium complex species present in a biological sample and preparing biological material therefrom, the kit comprising:
the device for collecting the biological sample according to claim 1, which forms a capturing scaffold; and
a container comprising a lysis buffer containing a solubilising agent, a detergent for simultaneously lysing the biological sample to make the biological sample available, inhibiting coagulation of the biological sample, and capturing at least a fraction of the biological material on the capturing scaffold.

21. (canceled)

22. (canceled)

23. (canceled)

24. (canceled)

25. The method of claim 10, wherein said parent polymer has had a flavouring agent added thereto to make collecting the biological sample more acceptable to the patient.

26. The method of claim 17, further comprising adding a reducing agent to said sample.

27. The method of claim 17, comprising elevating the temperature of the sample to 92° Celsius.

28. The method of claim 18, further comprising washing the capturing scaffold with de-ionised water.

29. The kit of claim 20, wherein said container further comprises a reducing agent.
